(19) United States
(12) Patent Application Publication
(10) Pub. No.: US 2005/0015102 A1
(43) Pub. Date: Jan. 20, 2005

(54) Hernia mesh-device with incorporated local anesthetic

(76) Inventor: Allen B. Chefitz, New Rochelle, NY (US)

Correspondence Address: Allen B. Chefitz, M.D., Suite 301, 140 Lockwood Avenue, New Rochelle, NY, 10801, US

(21) Appl. No.: 10/784,535

(22) Filed: Feb. 23, 2004

Related U.S. Application Data
(60) Provisional application No. 60/448,510, filed on Feb. 21, 2003.

(51) Int. Cl. A61B 17/08

(52) U.S. Cl. 606/151

(57) ABSTRACT

A mesh-device for surgical repair of a hernia which includes a local anesthetic. The local anesthetic may be either embedded within the mesh, or provided in reservoirs, but is generally evenly distributed across the mesh-device. The local anesthetic is released over a predetermined duration at a predetermined rate.

### CROSS-REFERENCE TO RELATED APPLICATION

This application claims priority to U.S. Provisional Application Ser. No. 60/448,510, which was filed on Feb. 21, 2003.

### BACKGROUND OF THE INVENTION

There are many types of mesh patches or devices used for the repair of hernia defects. Examples of mesh-devices include, but are not limited to, polypropylene mesh (Marlex, Bard's Perfix Plug & Patch, Ethicon's PHS, USSC's Surgipro), PFTE (polytetrafluoroethylene), Alloderm (Lifecell), and Surgisis (Cook). Since the introduction of mesh materials in hernia repairs, the recurrence rates have decreased to acceptable levels. Postoperative pain, however, remains a considerable problem after hernia surgery. With recurrence rates being low, postoperative pain accounts for most of the morbidity associated with hernia repairs.

### SUMMARY OF THE INVENTION

The hernia mesh, of which there are several types, contains local anesthetic equally distributed along its surfaces. After being placed into the patient's body space, the mesh releases a local anesthetic from reservoirs within the mesh at a nearly constant amount per unit time. The anesthetic is released for a predetermined duration, thereby reducing or eliminating any postoperative pain. The reservoir can be composed of membrane materials, such as ethylene-vinyl acetate copolymers, that control the rate of anesthetic delivery. The anesthetic can also be contained in an acrylic based polymer adhesive with a resinous cross-linking agent to provide a continuous source of anesthetic. The anesthetic can also be distributed evenly within the mesh material itself, not contained in reservoirs and released as described above.

### BRIEF DESCRIPTION OF THE DRAWING

FIG. 1a shows a top-view of a mesh-device containing reservoirs of local anesthetic.

FIG. 1b shows a bottom-view of a mesh-device containing reservoirs of local anesthetic.

Fig. 1c shows a mesh-device with embedded anesthetic within the mesh material.

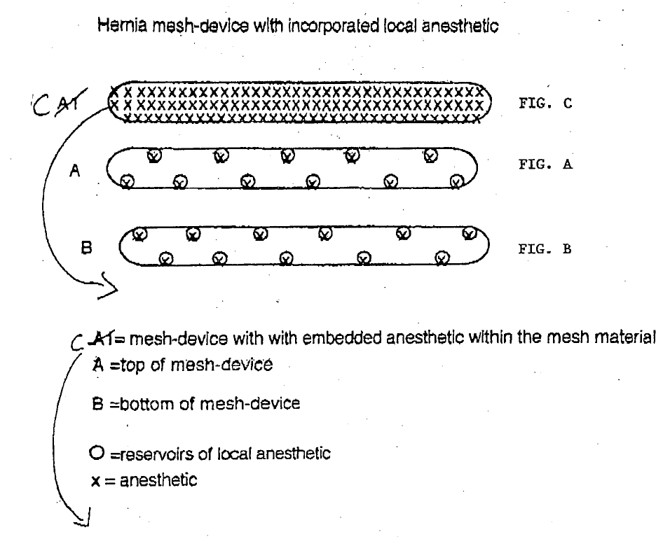

### DESCRIPTION OF THE INVENTION

This invention involves a mesh-device used to surgically repair a hernia. In particular this mesh contains a local anesthetic medicine embedded within the mesh, to be released post-operatively for maximal postoperative analgesia. Examples of local anesthetics include, but are not limited to, lidocaine (xylocaine), bupivocaine (Marcaine), and ropivacaine (Naropin).

1. A mesh-device used to surgically repair a hernia, comprising a local anesthetic and a mesh patch or prosthetic device.
